SEQUENCE LISTING (10 sequences)

SEQ ID NO: 1 - DNA, 3624 bp, Homo sapiens
cttagaccttggttagatttgaaccaaggccctcctcggcctcccaaagtgctgggattacaggcgtgagccactgcccagctgaaaattgatacatttttcaaatgagaagtatttttttcaaatgggtgttggaaagtgagtttcctcacattgttcatgttgtgacactaggttggtgctatgtaatgacatggatatgaacgagctgcctacgaacctccccgtggacactgtgaagcttcgcatagagaagactgtcatccgcagaatctctgcggaggccttctattacctggtggagctccagtatctctgggtgacttacaattccgtggccagcattgaccccagcagcttttacaacctgaagcaactgcatgagttgcgcttggatgggaattctctggctgctttcccttgggcatctctgctggacatgccccttctgaggaccctggacttgcacaataacaaaataaccagtgtgccaaatgaggcgctcaggtatctgaagaaccttgcctacttggatttatcaagcaacagactcaccacattgccaccagatttcctggagagctggactcatttagtttcaacaccttctggagtcctggacctttccccaagcaggattattcttggtctacaggacaatccttggttctgtgactgtcatatttccaaaatgattgagttgtcaaaggtcgttgaccctgctatagtgcttctggatccactgatgacttgcagtgaacctgagcgcctcacaggaattttgtttcagcgggctgaattggagcattgtctgaaaccatcagtgatgacctcagccaccaaaatcatgtctgctctgggcagtaatgttctactgcggtgtgatgccactggcttccccaccccacagatcacatggaccagatctgacagctcgccagttaattatacagtaatacaagaatctccagaggaaggagtcagatggtccataatgagcttgacaggcatttcttccaaagacgctggggattacaaatgtaaggccaaaaatctggctgggatgtcagaagctgtggttactgtgacagtgcttggcattaccacaactccaataccaccagacacttctgaaagaactggagatcatcctgagtgggatgtccagccgggatctggaagatctacatctgtatctagcgcatcatcatatctttggtcctcttccttctcccccacatcttctttttctgcttctactttgtctcctccctctactgcttccttctctttatctcctttctcctcctccactgtttcttcaaccacaactctgagcacaagcatctcagcaagtaccaccatggccaacaagcgatcattccagctccaccaaggtgggaaaagaaatttaaaggtggcaaagaatggaagtaagcttcctccagccagcacaagtaagaaagaagagctggcattgttggatcaaacaatgcttacggagacaaatgccgcaatagaaaacctcagggtggtcagtgagactaaagagagtgtgacattgacgtggaatatgatcaacaccacacataactctgcagtgactgtgttgtattccaagtatggtgggaaggacctgctgctgttgaatgcagactccagcaagaaccaagtaaccatagatggcttggaacccggtgggcaatacatggcctgtgtctgtccaaaaggagtgcctccccagaaagaccaatgcatcaccttttctactgaaagagttgaaggagatgattctcaatggtctctccttctcgtggtgaccagtactgcctgtgttgttatcttaccattgatttgtttcttgttgtacaaagtttgcaaactgcaatgtaaatcagaacctttttgggaagatgatttggcaaaggagacttatatccaatttgagaccctgtttcccaggtctcaaagtgtaggtgagctctggacacgaagccacagggatgactcagagaaattgctgctttgttctaggtcaagtgtggaatctcaggtgacttttaaaagtgaaggttccagaccagagtattattgctaaggttctgcagctcaggtgcatgtgagctacaaaactagcatctaagggtataattgaccctaggtttggatgacttttggacagactttcacattgtacatgaaaatcacaaatggaatgcttttaagtatgtttaaaaaataccatgagacctctgaactgaaaagacaaataatgttgattttttttcttgtgtggaaaaggtctacagaaataatttttagagtggtgcttaataaattattaatactttttagggtaatgttttagttcttaaaaataagttcatgtgaaagtagcaagtgaactctgtatttaaaaatatagttttttgagtcatgaggaaacattagcaaaaccagctaagccttgatgtttattcatgttcgaagtgcattatagcatttgctgtggatgttttctgacattgcctaatggagagcgtgggatcataaaggattttgtgttcactaaggttaaatctaactcacgggagagagagggctctgtctcttggtaccattatgcattgtgatactatagagttgtgttcatatttcttgtaagaaaaatttttgcttttctttagaaatataggtattagcaaaagtaagtatacaaagaggattatgacaaatagaccatttctttaaaaatggtaactgttgcatttaaacacatcgactgacagattatatggatatttaaaaataactttaaatcaatcttttaatattttgctgtttgctcttatgttctcccttctttcttccttccttgtcttccctccctttccttccttcattctttctttcttctgctctgtcagaagatatacaagtagccaaagcatcacgagccaagctgtttgtcttttgccatattgtcttttgattctcttgataaagagaaagcttttgaaattacttactgccctttgaaagtctgttttacaaccccaaaggatattttaggactttaaaagggaagcttcagtggattcatttggttagattcttttcctaggttaaaaacatcggggaacttaaatacataggaaaggagttcacaaataaaatttagtttttttagaaagagaatattttaataactgtagtatactacaagaaacatctcatggctttaagattaacgatagcgataaggcccactcaagcatctcacagtggtgatattacatgtcacttagcaggatgcaacctggcctagggatttccagaagacaaggccacctcagcacagatgcaactttcataaattttagaacaaagcctaaccttactagattaaacttgctctatgaaagaaacacttggtaattgacccagactgaatacatgtataagaaatgggaaagatctcccaaactctgagaacggtcctcaaatagaagctctcctagtcagttggtcatctgacctctgactatatccggcctatgacaccagccagctcctgctttctgtcttgtaagagcactgctggaataaactacttgagcatta

SEQ ID NO: 2 - DNA, 4999 bp, Canis familiaris
tgtcagactcatcaagcaatgcccgggacgagtattttaccaatggatgtatctcaaaacccactggcttctggaagaaactgaaataagagaaaaaaggagcgatgcatctctcagcatgtctgtgcattgtgcttgttagctttttggaaagagggagctgttcatgtccttcacagtgtacctgcgattatcatggcagaaatgacggaacgggatcaaggtcggtgctatgtaatgacctggatatgaatgaggtccctatgaacttccccgtggacactgtgaagctacgtatagagaagacagtcatccgcagaatccccgcagaggccttctactacctggtggagctccaatacctctgggtgacttacaattccgtggccagccttgatgccagcagcttttacaacctaaagcagctgcatgagttgcgcttggatggaaattctctggttgctttcccttgggcatctctgttggacatgccccatctgaggaccctggatttgcacaacaacagaataaccagtgtgccaaatgaggtggtcaggtacctgaagaaccttgcctacttggatttatcaagcaacaggctaaccacactaccaccagatttcctggagagctggtcccacttacttacaacatcattacgaagcctggaccttacaccacgaagaattattcttggtctgcaggacaacccttggttctgtgactgtcacatttccaaaatgatcgagttgtcaaaagttgctgacccttctatagtgcttcttgatccactgatgatttgtagcgaacctgagcgcctgacagggattttgtttcagcgggctgagttggagcagtgtctgaagccatcagtcatgacctcggccacccagatcacatctgctctgggtagtaacgttctgctgcggtgtgatgctactggctaccctaccccacagctcacgtggaccagatctgacagctcaccagttaattatacagtaattcaggaatctccaggggaaggagtcagatggtccataataagcttgactggtatttcttacaaggatgctggggattacaaatgtaaggccaaaaatttggctggaatgtcagaagctatggttactgtgacagtggttggtgttgtcacaaccaccatatcatcagagacctctgaaagaactggagatcaccctgaggaagaagtccagccaagatccagatctacatccccgcctggttcattgtcatcttcttggccttcctcctcccctgcttcttccatttttctttctaattctacttcatctcctcccccctctgctttcttctccttatctcctttcctctcctctattgtttcttcaaccacagctagaaacactggaatttcaacaagccccaccatggccagccagcagtcactccagctccacccagatgggaaaagaaatttaaaggtggagttgggtggaagtcagcttccttcaggtagtgcaagtagaaaagaagagttggcattgttggatgaagcaacgccagtggaaacaaataccacaatagagaatctcagggtagttggtgagactgaagagagtgtcacattgatgtggaacactgtcaacaccacacaaaagtctgagatgacagtgttgtactccaagtatggtgaaaaggacctgctgctgctgaatgctgactccagcaagaaccaagtaaccatagatggcttggagcctggtaggcaatatatagcatgtgtctgtccaaaaggaatacctccccagaaagaccaatgtattaccttttctactgacagagttgaaaaggaaggtgattctcaagtgtctttccttgttgtggtgagcggtaccgcctgtgttgttgtcttgccattgatatttttcctgttgtacaaagtttgcaaactgcaatgtaggtcagaatctttctgggaagatgacttggcaaaagaaacttatatccaatttgaaaccctgtcccccaggtctcaaagtgtaggggagctttggacgcgaaggcacagcgatgattcagaaaaattgctgctttgttctaggtcaagtgtagagtctcagatgacttttaaaaatgaaggtttgagatcagagtattattgctaagattttacacatgaaccccacaaaatttcttccatgcaactaaggattgaggatatagtcggccttctgtttggatgaccttttgttcaggtgtggttttctttttttcttttctttttttttttagataccatgtgaggggcacctggtttcctcagcctacaacttttgatcttggagttgtaagtttaagttcaagccccaattggatgtagagattacataaaaactaccatgtcatttctgaatcaaacaaacaaatagtcttgatttttgttgtgtggaagatatccatagaaatagtttttaggatagtgcttgataaattaatagtatgttttagggtagtgttttagtttttcaaaataaattcatgtgagaatagttataaaaaagtgaagtctgaattttgaaaagagttttctaagaagtgaagaaagattagtataaccagctaagccttgatgttcattcatgtttgaagtgctttatgacatttgctatgggtatttactgtcattgcctagtgggcaagagtgagaaagtacagaatttttcattcactaaggttaaatccaacccagtggagagggagggctccaactcttggtaccattatgcattacaagactgtatggttgattaatattgcaagaaaaaaaattttttatccttagaaatatagatactagcaaaaaagtttaaaaagaggatcctgaaaatagtaaggccatttctaaaaatagtaaccattgcatcctaatacatttgccaacagactatatggatctttttttaaaaaaatgttaaaaatttttttaatttaaaaaatttaaattttgaaaaaaattttgaaacaatctctttaatattgtgatatttacttttctcttctgctctgtttctccttctctctctcccctttccttccttcatttcttttttttctcttctgctttgtcaggagatattcaggtagccataggaccacagctaaactgtttgtcttttgccacgttatattttgatttcctggctaaagggaagaactttgagtcttttttagatctgaaaaagatgttttaggatttttttggtcaaattttctataaaaaacagagaaattgaactggagtggacatagataggaaaggggattcatacataaaatttaggtttttaaaagaaaagtttatcaccacctttactaaagattatattctctttacattggagattctggactacagaacagatttataaaggtgtgtcaggagtcctgaaatactctgaatagcaagttctaagaaatcctaaattttacataagcaatagattgacttcaatgttacacattgtggaaattataatacaagcaactggacaatgaaaaatcattcattccaaatggctgttaattaaattctagtttagacatattctgagtattagaaggagaccagcatggatggatggcctgtctagaactcccaaatgtccctttcttgtataatttcctctcctctttcctttccaccctgtttgtccttggtgctttcatcactgagtgcatttctaatcatctaatagttttaagttcagtattcattgttaagatgtgttagaaaaatattttttacaaacaattttgtgcctagcttcaaattctgttgggtcaacttaaaagatcgcaaaaactcacatttctttactctaaagacatcgtggaattcactgctacatttaaaaggttcatataggggtacctgggtgactcagtggttgagtgtgccctcagcttaggtcaagtcttgcatcaggctctctgcagggaacctacttctccctctgcttatgtctctgcctccctctctatgtctctcatgaatacataaataaaatcttttttaaaaaattaaaggttcatataaacaggctgccaaaatactaccacattagaaataatgactataggaaaaggtcacagaccttacctgaggtaagtggcacccaagttgtaaagaaaggagacttggcccaccctgatgaggtaaattacagactgccctcataaggaaatcaagtgaggcctgcacttaattttgaccaatgaggaaaatctagctgtgggtatgggtgggagaaagcacttctgtcgtcttgaacttggtcttggcccatgaagtgagtcctaagtaatgtgttgctaattgtgaaagaactgaaatagtaaattagctactctctaaaaatacacatatatgtatgttgacaaggttattctgatgatatcattgctcttacattcttacaagtatactttcctgagtggaagaaaaaggttgagcattgtggaattctagatcatcaatagcctatttatcaatgtcaccataagcattgtagagagcaataataattgatatatagccaatatatgtgacaattaaatatgattacagtgttttactctcagacaagatatatttgaatttgtaaaacttgggtaaatgatgagggtgtgtcacatatgagactatcataaatgatgtgttctgacataaaaaatgttgagaactgcacttatgattgctacaaaagagaaccctggactttagagtcagacaagtgctctggactttggggaaacttttacaaaatgttcaaagacaaaaaattttgacccacagacttgagtctcaaaaacagattatggttaacaaagggagattttaaaaacccaaaaacttttaagtgaataattgtggatttatttatttatttatttatttatttatttatttatttatgattttatttatttattcatgagtgacacacagagagag

SEQ ID NO: 3 - DNA, 5008 bp, Equus caballus
tgcaagatacacttgaaggttaagtaactacagatctcagctaatcccgtgtaatctgctgttggtaaatggctgtttacggtcctggcacaccagcttctgaatgcttaggattttgttttcaccttttgttcaaataacctctgaaaggagcgatgcgtctctttgcatgcctgtgcattgtgctcagctttttggaaagagtgagctgttcgtgtccttcacagtgcacctgcgactactggggcagagacgatggctcgggatcaaggttggtcctatgcaatgacctggacatgaacgaggtccctacgaacttccccgtggacgccgtgaagcttcgcatagagaagacagtcatccgcaggatcccggccgaggccttctactacctggtggagctccagtacctctgggtgacttacaattccgtggccagccttgatgtgagcagcttttacaacctgaagcagctgcatgagttgcgcttggatgggaattctctggctgccttcccttgggcatctctgctggacatgccgcttctgaggaccctgggtctgcacaataacagaataaccagtgtgccaaatgaggccctcaggtacctgaagaaccttgcctacttggatttatcaagtaacagactaacaacgctgccaccagatttcctggagagctggtctcacctagttgcaacatcatctagaagcctggccctcacaccaagaagaattattcttggtctgcaggacaacccttggttttgtgactgtcacatttccaaagtgatcgagttgtctaaagttgctgaccccgctgtagtgctcctcgatccactgatgatttgtagtgaacccgagcgcctggcagggattgcatttcagcgggctgagctggagcagtgtctgaagccatcggtgatggcctcggccacccagatcacgtccgctctgggcagtaacgttctgctgcggtgcgatgccaccggctaccccaccccacagctcacgtggaccagatccgacagctcgccagttaattatacagtaattcaggaatctccagaggaaggagtcagatggtccataataagcttgacaggcatttcatacaaggatgctggggattacaaatgtaaggccaaaaatttggctgggatgtcggaagctgtggttactgtgacagtggttggtgttgtcacaaccaccatatcatcagacacttctgaaagaggaactggagatcagcctgagcaagaggtccaggcagactctagaactccatccatacctggcccatcggcatctccctggcctccttcctccgctgcttcttccacttttctttctacttctactttgtctcctccctccactgcttcctcctccttatctcctttcttctcctcctttgtttctccaaccacaactcgaagcactagaatttcaacaagcaccaccgtggctggccagcagtcactccagctctacccagatgtgaaaagaaatttaaaggtggagatgggtggaggtaagcttcctcctgctagtgcaagtaaaaaagaattggcgttgttggatcaagcaatgtctatggagatgggtgccacaatagaaaatctcagggtggtcagcgagaccgaagagagtgtgactttggtgtggaacaccatcaacagcatgcgtgactctgaggtgaacgtgttgtattccaagtatggggagaaggacctgctgatgcttaatgctgactccagcaagaaccaagtaaccatagatggcttggagcctgggaagcaatacatagcatgtgtctatccgaaaggagtgcctccccggaaggaccagtgcatcaccttttctactaggacggttgaagaagtgggtgattttcaagggtctttccttatcgtggtgagcggtactgcctgtgttgttgtcttgccattgatatttttcctgttgtacaaagtttgcaaactgcaatgtaggtcagaatccttttgggaagatgatttagccaaagagacttacatccagtttgagaccctgtcgcccaggtctcaaagtgtaggggaactttggactcgaaggcacagagacgattcagaaaaggtgctgctttgttctaggtcaagtgtggaatctcagatgacttttaaaagcgagggttctagaccagactattattgctaaggttttacagctcaggtgcatgtgaatttcttccaattgaggatctgagggtatagttgaccgtctgtttggatgacttttggacagactttcccatccttcatgaaaatcacaaatggagtgcttttaattgtgtttaaaaaaataccatgagacttctgaactgaaaggacaaataatcttgactttttttgtgtggaaggtgtccatagaaataatttttagagcagtgcttaataagttattaatatgttttagggtaatgttttagtccttaaaaataaatttatgtgaaaatggttgtaaaaaagtgaaatctgaattttaaaaagatagttttttgggcagtgaagaaagattagtgtaaccagctatgccttgacttcactcctgttcaaagtgccctgtggggtttgctgtggatactggctgccattacctggtgggcgagagtggaagagtaagttttgcattcgctaaggtgaaatccagccccgtggagaaggagggctccgtctcttcgtgcccgtaggcattacaatactgtgtagttgtattcatattgcaagtaagaaacagttttcccatgtcctaagaaacataggtattagcaaaaataagtttagaaagaggatcatgatgcatagtaaggtcatttcaaaaaatagtaactattgcattctagtacatttgctaagtgactgtatgggtctgtttttaaaccatggtgctccttttaaaacaatctcttgaatattttgatatttactcttcttctctccctccgctcttccttccgtcgtttcttttttccccttctgctttgtcagaagatattcaggcggccaaaggaccacagctaagctgtttgtcttttgccatgttattttgatttcctggataaagggaagaacttggagagtcttttttagagccaaaaggcatgttttaggattttgaaagattatcttcagtggatgcatttggtcagactttgtaggttagaaacatcaaggaaattgaattggagtggccagatgaatgggagaaaaggagcgtcatagataaaatttagttttttttttaaaaaggagtttttcaccacctttactcaagattatattccctttacattgaagattttggagtatagaacagatttataaaggtatgtcagaagtcctgaaatatcctgagaagcaagttctcagaaaatactatgtttggtaagaaacagattgtctttgatgtaacacgttgtggaaattataatacaagcagctgcagaatgagaaattgtttattccaaatggctcttaattaaaaagaaaaaagaatttcagtttagccataatccgcacatcagaagatgaccagcatggatggatggcctgctctgaactcccccacttcccctgctggtataatttcctctcctttccagcttttttgtccttggtgcttccatcactgagtgcattatctaatgagctaacagtttttaattcagtattcattgttgggatttgttagaaaaatatattttgcagacaattttgtgtttagcttcaaattattttagatcaacttattttaaactaaaagattgcaaaaaccttcatttctctactctaaagatatcatgaaatttcattgccatatttaaagggctctgccaaaatactgcgggagaatgttacagactactgagctaaatggcacccaaattgtacaaaggcaggttatttcaaaaaaggagacttggaccatcatgatgagataagttactgactgcccttataatcaaaatcaagtgaggtctggacttaattttgaccaatgagaaaaagctagttggtgacatggaggggagaaagcacttttgtcatcttcaacttggttttagcacaggaggtgaattgcaagtaatttgttactaatagtgaaagaatataattgtaagtcatctgctttctaaaaatacatagatttgagtctatgcaaggttatcctggtaatatcattgctctcatgttcttacgagtacgctttcttgagtgggagagaaagactgacatgctagcaagcagcatggaattccacgtaatcaacagtctgttaatcaatgtcaccataagcgttgtggatgatatatagcataactgatattgagccaatatatgttgcaattagatataattacaatgtcttactttcagacaaaatatattggagatttgtaaaagttggggtacacggtgagggcgtgttccacgtgagactatcatcagtgatgtgctatgacataaaaaatgttgaggactccaaagtaatagtttatgatggccggaaaagcaaaccgtggactttggagtcagataagtgccctggactttgggaaacttttatgaaagtttcaaagagaaaaatttttgacccctaggcttgagtctcaaaaaggcattatggctaatgaagggagattaaaaaagaaatttccagatgaatgattgtgaaataattcaaataaaatgggagacagctcaggaaagttgaatggctgtctagtaacttaaaatttcaaatgataagtataaaggaaagaaagaagtttgagtaaccaatgacgaatgtagaaatgtagcaaatctattataaaagtgtcaaaaatttagaccacccaataaattaaggatcaaaaagaaagggtaaagttgaccagaaggaactataaggcttgttttttgttttgttttgtttcagtggaaagagaatgctaatgtttgtattgataagtagacttttctttt

SEQ ID NO: 4 - DNA, 2046 bp, Mus musculus
atgtggctctcggcctgcctgtgccttgtgcttagcttcctgggaggagtgaacggtacctgcccttctcagtgctcctgtgagtatcacggcagacatgacggctcaggatcaaggttagtgttgtgtaacgacctggatatgaacgaagtcccagcaaacttccccgtggacacctcgaagcttcgcatagagaagactgtggtccgcaggctccccgccgaggccttctactacctggtggagctgcagtacctctggctggcttacaactcagtggccagcatagaaaccagtagcttctataacctgaggcagctgcacgagttgcgtttggatgggaattctctgaccgctttcccttgggtgtctctgctggacatgccccacctgaggacgctggacttacacaataacagaatagccagtgtgccaaacgaggcggtcagatatctgaggaacctcacctgcttggacttgtcgagcaaccgactgaccacgctgccaccagatttcctagacagctggtctcatttagccgtgacaccgtctagaagcccggactttccaccgagaagaattattcttggtttgcaggacaacccctggttctgtgactgtcacatttccaaggtgatcgagctgtcgaaagtcaccgaccacgctgttgttcttcttgatcctctgatggtctgcagtgaacccgagcgcttccaaggaatcttgttccagagggtagagttggaaaagtgtctgaagccgtccgtgatgatgtcagctaccaaaatcacatctgctctgggtagtaatgttctgctgagatgtgatgccaagggtcaccccaccccacagctgacgtggaccagatccgacggctccacagttaactatacagtaattcaggagtctccaggagaaggcatcagatggtccatcataagcttgaccagcatctctcacaaggatgctggggattacaggtgtaaagccaaaaatctggcagggatttcggaagctgtggttactgtgacagttgttggtggtgttacgaccaccctatcaccagacagttcagaaagaagtcctggagagccccctgagcagcatccccagccaggattaggaggatcaacacctccatctaaatcctggttatcacccgggctcacttccgctccttcctaccccaccccctcagcagctctctatacatctacgtggtcccctcctccctcctccttgcctcccatcttctcagctgcttctgcaaccaccagtgtacaaaccagcatctcaggacgcaccgccaggactagccaccagccacccctgctccaccccggtgggaaaagcaatgcgaagatagagaagaacggaaggaagtttcctccgctcagcgcaagtaagaaagaagagttggcgttgttggatcaggcagcgccaatggaaacaaacgtcactatcaaagacctcagggtggcccgtgaaaccggcgtgagtgtgactctgatgtggaacagcagcagcagcacacaagagtcctctgtgactgtgctgtattctaagtacggtgagaaggacctgctgttagtgaatgcagacgactatggcaagaaccaggcaaccataaatggcctagagcccggaagtcagtatgtggcatgtgtctgtccaaaaggagtgggtccccgggaggatctgtgtatcaccttttctaccaacagagttgaggggcgtggctcacagtggtcattgctcctcgtggtgaccagtactgcctgtgttatagtcgtgcccctaatttgtttcttattatataaagtctgcaaattgcaatgcacatcggaccctttctgggaagaggatttgtcaaaagagacatatatccaatttgagaccctgtcacccaggtcacagagtataggggagctctggacccggaggcacagagacgatggcgagaggttgctgctatgctcccagtcaagtgtggactcccagatgaatcttaagagcgatggctgtaggacggagtattatggctga

SEQ ID NO: 5 - DNA, 301 bp, Unknown
  GRK1
ctagtgggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccgggctcccaggggcttcccagtggtccccaggaaccctcgacagggccagggcgtctctctcgtccagcaagggcagggacgggccacaggcaagggcgc

SEQ ID NO: 6 - DNA, 413 bp, Unknown
  GRM6
gatctccagatggctaaacttttaaatcatgaatgaagtagatattaccaaattgctttttcagcatccatttagataatcatgttttttgcctttaatctgttaatgtagtgaattacagaaatacatttcctaaatcattacatcccccaaatcgttaatctgctaaagtacatctctggctcaaacaagactggttgctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaa

SEQ ID NO: 7 - DNA, 130 bp, Unknown
  5′ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 8 - DNA, 130 bp, Unknown
  3′ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 9 - DNA, 542 bp, Unknown
  WPRE
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcg

SEQ ID NO: 10 - DNA, 215 bp, Unknown
  polyA
gcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

(19) United States
(12) Patent Application Publication
(10) Pub. No.: US 2024/0092858 A1
(43) Pub. Date: Mar. 21, 2024

(54) TREATMENT OF CONGENITAL STATIONARY NIGHT BLINDNESS USING GENE THERAPY

(71) Applicants: SORBONNE UNIVERSITE, Paris (FR); CENTRE NATIONAL DE LA RECHERCHE SCIENTIFIQUE, Paris (FR); INSTITUT NATIONAL DE LA SANTÉ ET DE LA RECHERCHE MÉDICALE (INSERM), Paris (FR)

(72) Inventors: Christina Zeitz, Paris (FR); Deniz Dalkara, Paris (FR); Juliette Varin, Paris (FR); Isabelle Audo, Paris (FR); Serge Picaud, Paris (FR); José-Alain Sahel, Paris (FR)

(73) Assignees: SORBONNE UNIVERSITE, Paris (FR); CENTRE NATIONAL DE LA RECHERCHE SCIENTIFIQUE, Paris (FR); INSTITUT NATIONAL DE LA SANTE ET DE LA RECHERCHE MEDICALE (INSERM), Paris (FR)

(21) Appl. No.: 18/255,951

(22) PCT Filed: Dec. 8, 2020
(86) PCT No.: PCT/IB2020/001097

§ 371 Date: Jun. 5, 2023

(51) Int. Cl. C07K 14/705; A61K 48/00; A61P 27/02; C12N 15/86

(52) U.S. Cl. CPC C07K 14/70503; A61K 48/0058; A61P 27/02; C12N 15/86; C12N 2750/14143; C12N 2830/008; C12N 2830/48

(57) ABSTRACT

The present invention relates to an expression cassette allowing expression of a functional LRIT3 protein in mammal eyes; said expression cassette is inserted in an expression vector, preferably an adeno-associated virus (AAV); accordingly, the present invention further relates to a recombinant adeno-associated virus (AAV) vector carrying a nucleic acid sequence encoding a normal LRIT3 gene, or fragment thereof, under the control of regulatory sequences which express the product of the gene in the ocular cells, a pharmaceutically acceptable composition comprising such a recombinant AAV vector and to its use for the treatment of congenital stationary night blindness

The present invention relates to an expression cassette allowing expression of a functional LRIT3 protein in mammal eyes; said expression cassette is inserted in an expression vector, preferably an adeno-associated virus (AAV); accordingly, the present invention further relates to a recombinant adeno-associated virus (AAV) vector carrying a nucleic acid sequence encoding a normal LRIT3 gene, or fragment thereof, under the control of regulatory sequences which express the product of the gene in the ocular cells, a pharmaceutically acceptable composition comprising such a recombinant AAV vector and to its use for the treatment of congenital stationary night blindness.

Congenital stationary night blindness (CSNB) is a heterogeneous group of non-progressive rare inherited retinal disorders (IRDs) (1). The most frequent type of CSNB is the Schubert-Bornschein-type, which is due to a disruption of the signal transmission between photoreceptors (PR) and bipolar cells (BCs). It can be further subdivided in two forms: incomplete CSNB (icCNSB) and complete CSNB (cCSNB) (1).

Patients affected with cCSNB are mainly characterized by impaired night vision, decreased visual acuity, high myopia, nystagmus and sometimes strabismus. cCSNB is a largely non-degenerative disease with normal fundi. Clinically, it can be diagnosed by full-field electroretinogram (ERG) recordings showing an isolated ON-pathway dysfunction (1). At low light intensities in dark-adapted (DA) conditions, the b-wave is absent, showing a transmission defect between rods and ON-bipolar cells (ON-BCs). With a brighter flash, the a-wave is maintained, representing normal rod and cone function, while the b-wave is absent. In light-adapted (LA) conditions, there is a typical square-shaped a-wave, a sharply arising b-wave with no oscillatory potentials and a variable but often decreased b/a ratio due to cone ON-BCs dysfunction (1). This is in accordance with the expression profile of the genes mutated in patients with cCSNB including NYX, TRPM1, GRM6, GPR179 and LRIT3 (1). Indeed, these genes code for proteins localized in the outer plexiform layer (OPL) affecting signal transmission between photoreceptors (PR) and ON-BCs.

Several animal models of cCSNB have been described, including Appaloosa horses with a TRPM1 defect (2), beagle dogs with an LRIT3 defect (3) and different mouse models lacking various genes implicated in cCSNB (1). All reveal a similar ERG in DA conditions, which resembles those of patients; while the a-wave is maintained the b-wave is absent. In LA conditions, the phenotype of Appaloosa horses with the Trpm1 detect and beagle dogs with the Lrit3 defect, resemble also the cCSNB phenotype observed in patients. However, in LA conditions all mouse models for cCSNB show a significant difference in the phenotype compared to human patients. In contrast to patients with cCSNB, mouse models reveal a complete absence of the b-wave in LA conditions.

The Inventors studied cCSNB due to mutations in LRIT3 (also known as CSNB1F or FIGLER4) that codes for the Leucine-rich Repeat Immunoglobulin-like Transmembrane Domain 3 protein and the respective mouse model (nob6 also called Lrit3^{-/-), (}5), Lrit3^{-/-} mice are characterized by the absence of LRIT3 in the OPL, lack of the ERG b-wave in both DA and LA conditions, altered optomotor responses in DA conditions and abolished ON-responses at the retinal ganglion cell level upon multi-electrode array (5, 6). Albeit the rote of LRIT3 has not been completely elucidated, they showed that LRIT3 is crucial for the correct localization of TRPM1 at the dendritic tips of all ON-BCs (7) and that it is a key actor of the cone synapse formation/maintenance (6). Recently it was suggested that LRIT3 is essential for the localization of nyctalopin, encoded by NYX, and that the loss of TRPM1 in mice lacking LRIT3 was due to the loss nyctalopin (8).

Several gene therapies for IRDs have been developed over the years (9). However, treatment for CSNB patients is yet unavailable. The most suitable treatment for cCSNB appears to be gene therapy. Indeed, cCSNB represents a non-progressive disorder, in which retinal morphology is well preserved (1), genes underlying this disorder have been identified and specific targeting of ON-bipolar cells in primate retinas with AAV-vectors has been demonstrated. Due to this stable non-degenerating condition treatment could be in theory administered at any time. However, recent findings revealed functional restoration mainly in very young mice. Indeed, a partial functional rescue of the b-wave under DA conditions using an intravitreal AAV-mediated gene replacement approach has been obtained in mice lacking Nyx, when treating mice at P2 and targeting bipolar cells (10). Similar observations were made in mics lacking Lrit3, when treating mice at P5 or P35 and targeting rod photoreceptors, with more significant restoration at P5. In addition to restoration of the respective proteins, TRPM1 was also relocalized (10, 11). However, restoration of the b-wave under LA conditions could not be obtained (5, 10, 11).

The Inventors have now been able to restore the production and function of LRIT3 in the adult nob6 mouse model under DA conditions by preparing an expression cassette allowing an efficient LRIT3 expression in mammal retinal cells.

The present invention refers to the following objects:
1—a expression cassette comprising (i) GRK1 promoter and/or a 200 bp enhancer of the GRM6 promoter fused to the SV40 promoter (GRM6 promoter) and (ii) a nucleic acid sequence encoding LRIT3 that is operably linked to said promoter.
2—The expression cassette of item 1, wherein said nucleic acid sequence encoding human LRIT3 has at least 90% identity with SEQ. ID. N°1.
3—The expression cassette of item wherein said nucleic acid sequence encoding canine LRIT3 has at least 90% identity with SEQ. ID. N°2.
4—The expression cassette of item 1, wherein said nucleic acid sequence encoding horse LRIT3 has at least 90% identity with SEQ. ID. N°3.
5—The expression cassette of item 1, wherein said nucleic, acid sequence encoding mouse LRIT3 has at least 90% identity with SEQ. ID. N°4.
6—The expression cassette of anyone of the preceding items, comprising only a GRK1 promoter, or only a GRM6 promoter or both GRK1 promoter and GRM6 promoter.
7—A recombinant expression vector comprising the expression cassette according to anyone of items 1 to 6.
8—A recombinant adeno-associated virus (AAV) vector comprising:
   an AAV capsid;
   an expression cassette according to anyone of items 1 to 6.
9—The recombinant AAV vector of item 8, wherein the AAV capsid is AAV 2.7m8.
10—The recombinant AAV vector of item 8 or item 9, wherein it also contains woodchuck hepatitis posttranscriptional regulatory element (WPRE) and/or poly-Adenine (polyA).
11—The recombinant AAV vector of anyone of the items 8 to 10, wherein the expression cassette is placed between two ITRs, preferably, ITRs from AAV2 serotype.
12—The recombinant AAV vector of item 10 or item 11, wherein it expresses LRIT3in photoreceptor cells.
13—The recombinant AAV vector of anyone of the items 8 to 12, for use as a medicament.
14—The recombinant AAV vector of anyone of the items 8 to 12, for use in the treatment of CSNB.
15—The recombinant AAV vector of any one of the items 8 to 12 for use according to item 14, wherein CSNB is cCSNB, in particular to correct impaired night vision, decreased visual acuity, high myopia, nystagmus and strabismus.
16—The recombinant AAV vector of anyone of the items 8 to 12 for use according to item 14 or item 15, wherein the amount of recombinant AAV vector administered is sufficient to provide a therapeutic effect to said mammal.
17—The recombinant AAV vector of anyone of the items 8 to 12 for use according to item 16, wherein said recombinant AAV vector is to he administered intraocularly, at between about 10⁹ to 10 ¹⁴ vectors/eye of the mammal.
18—A pharmaceutical composition comprising a recombinant AAV vector according to any one of the items 8 to 12 and a pharmaceutical acceptable carrier.
19—A pharmaceutical composition according to item 18, comprising (i) a recombinant AAV vector according to any one of the items 8 to 12 comprising she GRK1 promoter and (ii) is recombinant AAV vector according to any one of the items 8 to 12 comprising the GRM6 promoter.
20—The pharmaceutical composition according to item 19 wherein the ratio of recombinant AAV vector comprising the GRK1 promoter to the recombinant AAV vector comprising the GRM6 promoter is comprised between 1:1 and 1:0.
21—The pharmaceutical composition according to anyone of the items 18 to 20, wherein the pharmaceutical acceptable carrier a liquid carrier, such as sterile, pyrogen-free water and sterile, pyrogen-free, phosphate buffered saline.
22—The pharmaceutical composition according to anyone of the items 18 to 21, for use in the treatment of CSNB.
23—The pharmaceutical composition according to anyone of the items 18 to 21, for use as in item 22, wherein said composition is for intravitreal or subretinal administration.
24—The pharmaceutical composition according to anyone of the items 18 to 21, for use as in item 22 or item 23, wherein the treatment is in a mammal, and preferably, said mammal is a human.
25—The pharmaceutical composition according to anyone of the items 18 to 21, for use as in items 22 to 24, wherein the amount of recombinant AAV vector administered is sufficient to provide a therapeutic effect to said mammal.
26—The pharmaceutical composition according to anyone of the items 18 to 21, for use as in items 22 to 25, wherein said composition is administered more than once.
27—The pharmaceutical composition according to anyone of the items 18 to 21, for use as in items 22 to 26, wherein administration of the pharmaceutical composition is repeated at least once in the same eye and/or contralateral eye.
28—A method of treating CSNB in a subject, said method comprising administering to said subject an effective concentration of a pharmaceutical composition according to anyone of the items 18 to 21.

The present invention thus relates to an expression cassette comprising (i) GRK1 promoter and/or a 200 bp enhancer of the GRM6 promoter fused to the SV40 promoter (further referred as the GRM46 promoter) and (ii) a nucleic add sequence encoding LRIT3 that is operably linked to said promoter.

The promoter is preferably chosen amongst GRK1 promoter and/or GRM6 promoter and may be derived from any species.

In one embodiment, the promoter is the human G-protein-coupled receptor protein kinase (GRK1) promoter (Genbank Accession number AY327580), in a particular embodiment, the promoter is a 295 nt fragment (positions 1793-2087) of the GRK1 promoter and is of SEQ. ID. N°5.

In another embodiment, the promoter is the 200 bp enhancer of the mouse Glutamate Metabotropic Receptor 6 (GRM6) promoter fused to the SV40 promoter thereafter GRM6 promoter) and is of SEQ. ID. N°6.

The expression cassette of the invention may comprise either (i) only GRK1 promoter, (ii) only GRM6 promoter or (iii) both GRK1 promoter and GRM6 promoter, in such embodiment, GRK1 promoter and the LRIT3 nucleic acid sequence may be placed before or after GRM6 promoter and the LRIT3 nucleic acid sequence. Preferably, the expression cassette comprises only GRK1 promoter.

The term “LRIT3” as used herein, refers to the full-length gene itself or a functional fragment as further defined below.

The nucleic acid sequence encoding a normal LRIT3 gene may be derived from any mammal which natively expresses the LRIT3 gene, or homolog thereof.

In another embodiment, the LRIT3 gene sequence is derived from the same mammal that the vector comprising the expression cassette of the invention or the pharmaceutical composition comprising it is intended to treat.

In another embodiment, the LRIT3 is derived from a human and is defined by SEQ. ID. N°1; accordingly, the nucleic acid sequence encoding human LRIT3 has at least 90%, preferably at least 95%, 98% or 99%, of identity with SEQ. ID. N°1.

In other embodiments, the LRIT3 is derived from:
canine and is defined by SEQ. ID. N°2, accordingly, the nucleic acid sequence encoding canine LRIT3 has at least 90%, preferably at least 95%, 98% or 99%, of identity with SEQ. ID. N°2;
horse and is defined by SEQ. ID. N°3, accordingly, the nucleic acid sequence encoding horse LRIT3 has at least 90%, preferably at least 95%, 98% or 99%, of identity with SEQ. ID. N°3;
mouse and is defined by SEQ. ID. N°4, accordingly, the nucleic acid sequence encoding mouse LRIT3 has at least 90%, preferably at least 95%, 98% or 99%, of identity with SEQ. ID. N°4;

The expression cassette of the invention may be inserted in any expression vector allowing ocular expression of functional LRIT3, said expression cassette is particularly efficient when cloned in an adeno-associated virus (AAV) vector.

Accordingly, the present invention also refers to an expression vector comprising the expression cassette according to the invention.

Said expression vector may alternatively be chosen among lentiviral vectors.

According to a particular embodiment, the present invention refers to a recombinant adeno-associated virus (AAV) vector comprising:
an AAV capsid;
an expression cassette as previously described, that is to say comprising (i) GRK1 promoter and/or a 200 bp enhancer of the mouse GRM6 promoter fused to the SV40 promoter (further referred as the GRM6 promoter) and (ii) a nucleic acid sequence encoding LRIT3 that is operably linked to said promoter.

LRIT3 expression is performed thanks to an expression cassette placed between two ITRs; said vector comprises a promoter, a nucleic acid sequence encoding LRIT3 and a terminator.

Unless otherwise specified, the AAV ITRs, may be readily selected from among any AAV serotype, including, without limitation, AAV1, AAV2, AVV3, AAV4, AAV5, AAV6, AAV7, AAV8, AAV9 or other known and, unknown AAV serotypes. These ITRs or other AAV components may he readily isolated using techniques available to those of skill in the art from an AAV serotype. Such AAV may be isolated or obtained from academic, commercial, or public sources (e.g., the American Type Culture Collection, Manassas, VA). Alternatively, the AAV sequences may be obtained through synthetic or other suitable means by reference to published sequences such as are available in the literature or in databases such as, e.g., GenBank, PubMed, or the like. Preferably, ITRs are from AAV2 serotype; for example, 5′ ITR is of SEQ. ID. N°7 and 3′ ITR is of SEQ. ID. N°8.

According to a specific embodiment, said expression cassette may also contain woodchuck hepatitis postranscriptional regulatory element (WPRE); for example, sequence of WPRE is SEQ. ID. N°9 and/or efficient RNA processing signals such as splicing and poly-Adenine (polyA) signals, in a preferred embodiment, it comprises polyA from human or bovine growth factor (bGH), for example of SEQ. ID. N°10.

AAV is a protein shell surrounding and protecting a small, single-stranded DNA genome of approximately 4.8 kilobases (kb). AAV belongs to the parvovirus family and is dependent on co-infection with other viruses, mainly adenoviruses, in order to replicate. Initially distinguished serologically, molecular cloning of AAV genes has identified hundreds of unique AAV strains in numerous species. Its single-stranded genome contains three genes, Rep (Replication), Cap (Capsid), and aap (Assembly). These three genes give rise to at least nine gene products through the use of three promoters, alternative translation start sites, and differential splicing. These coding sequences are flanked by inverted terminal repeats (ITRs) that are required for genome replication and packaging.

Recombinant AAV (rAAV), which lacks viral DNA, is essentially a protein-based nanoparticle engineered to traverse the cell membrane, where it can ultimately traffic and deliver its DNA cargo into the nucleus of a cell. In the absence of Rep proteins, ITR-flanked transgenes encoded within rAAV can form circular concatemers that persist as episomes in the nucleus of transduced cells.

More than 30 naturally occurring serotypes of AAV are available. Many natural variants in the AAV capsid exist, allowing identification and use of an AAV with properties specifically suited for ocular cells. AAV viruses may be engineered by conventional molecular biology techniques, making it possible to optimize these particles for cell specific delivery of LRIT3 nucleic acid sequences, for minimizing immunogenicity, for tuning stability and particle lifetime, for efficient degradation, for accurate delivery to the nucleus, etc.

Thus, LRIT3 overexpression can be achieved in the retinal cells through delivery by recombinantly engineered AAVs or artificial AAV's that contain sequences encoding LRIT3.

In one embodiment, the AAV capsid AAV2.7m8.

An example of a recombinant AAV vector comprising GRM6 promoter according to the present invention is illustrated in FIG. 1.

Inventors have shown that the rAAV vector according to the present invention allows the protein localization of a functional LRIT3 in the outer plexiform layer, between both rod and cone photoreceptor cells and ON-bipolar cells.

The present invention also relates to the recombinant AAV vector of the invention for use as a medicament.

The present invention also relates to the recombinant AAV vector of the invention for use in the treatment of CSNB in a mammal, said CSNB may result from a LRIT3 defect; in particular the recombinant AAV vector of the invention is used in the treatment of cCSNB.

Such treatment may improve impaired night vision and/or increase visual acuity.

For such treatment, the amount of recombinant AAV vector administered is sufficient to provide a therapeutic effect to the mammal to be treated.

An effective dose of the expression vector according to the invention to he administered is between about 10⁹ and 10¹⁴ vectors per eye.

It is desirable that the lowest effective concentration of virus be utilized in order to reduce the risk of undesirable effects such as toxicity, retinal dysplasia and detachment. Still other dosages in these ranges may be selected by the attending physician, considering the physical state of the subject, preferably human, being treated, the age of the subject, the particular ocular disorder etc.

According to another embodiment, the present invention related to a pharmaceutical composition comprising a recombinant AAV vector according to the present invention and a pharmaceutical acceptable carrier.

The pharmaceutical composition according the invention may comprise either (i) a recombinant AAV vector comprising the GRK1 promoter, (ii) a recombinant AAV vector comprising the GRM6 promoter, a recombinant AAV vector comprising the GRK1 promoter and the GRM6 promoter or (iv) a recombinant AAV vector comprising the GRK1 promoter and a recombinant AAV vector comprising the GRM6 promoter, in this last embodiment, the ratio of recombinant AAV vector comprising the GRK1 promoter to the recombinant AAV vector comprising the GRM6 promoter is comprised between 1:1 and 1:0.

Pharmaceutically acceptable carrier suitable for administration to the eye, e.g., by intravitreal subretinal injection, are buffered saline or other buffers, e.g., HEPES, to maintain pH at appropriate physiological levels, and, optionally, other medicinal agents, pharmaceutical agents, stabilizing agents, buffers, carriers, adjuvants, diluents, etc.

For injection, the carrier will typically be a liquid. Exemplary physiologically acceptable carriers include sterile, pyrogen-free water and sterile, pyrogen-free, phosphate buffered saline.

In one embodiment, the carrier is an isotonic sodium chloride solution.

In another embodiment, the carrier is balanced salt solution.

In one embodiment, the carrier includes tween. If the virus is to be stored long-term, it may be frozen in the presence of glycerol or Tween20.

The present invention also relates to said pharmaceutical composition for use in the treatment of CSNB in mammal, in particular in the treatment of cCSNB.

Administration of the pharmaceutical composition may be made by different routes; preferably, it is injected via intravitreal or subretinal administration.

The pharmaceutical composition may be delivered in a volume chosen by the person skilled in the art depending on the mammal to be treated, the size of the area to be treated, the viral titer used, the route of administration, and the desired effect; usually such volume may be comprised between 1 to 150 μl.

The effective concentration of a recombinant AAV vector formulated in the pharmaceutical composition is as described above.

The pharmaceutical composition of the invention is preferably for the treatment of any mammal in need of such treatment, including particularly humans. Other mammals in need of treatment include dogs, cats, or other domesticated animals, horses, livestock, laboratory animals, including non-human-primate etc. The subject may be male or female.

The pharmaceutical composition of the invention may be administered according to a regimen chosen by the person skilled in the art depending the disease to be treated, for example, administration may occur once or more than once. It may be at least once in the same eye and/or contralateral eye.

The present invention related to a method of treating CSNB in a mammal subject, said method comprising administering to said subject an effective concentration of the pharmaceutical composition of the invention. Preferably, the pharmaceutical composition is injected in eye, via intravitreal or subretinal route.

### DESCRIPTION OF THE FIGURES

FIG. 1: Schematic representation of a recombinant AAV vector according to the present invention.

FIG. 2: Schematic representation of the cellular targets of the three constructs.

PR-Lrit3 construct targeting both, rod (light grey) and cone (dark grey and black) PRs circled in black, the BC-Lrit3 construct targeting both, rod (dark grey) and cone ON-BCs (light grey) both circled in dark grey, and the OPL-Lrit3 construct targeting both, rod and cone PRs and ON-BCs by co-injection of the PR-Lrit3 and BC-Lrit3 constructs, circled in light grey.

FIG. 3: Localization of LRIT3 and TRPM1

Representative confocal images of cross-sections centered on the OPL (black space in the middle of two rows of grey nuclei) of Lrit3^{+/+}, Lrit3^{-/-}, Lrit3^{-/-}-BC-Lrit3, Lrit3^{-/-}-OPL-Lrit3 and Lrit3^{-/-}-PR-Lrit3 retinas stained with an antibody against LRIT3 (grey dots, left row) and TRPM1 (grey dots, right row). Arrow heads point to the synapse between cone and cone-BCs and arrows point to the synapse between rod and rod-BCs. Scale bar, 10 μm.

FIG. 4: ERG recordings.

A) Representative scotopic ERG traces at 2 months post-injection for Lrit3^{+/+} (dotted black line), Lrit3^{-/-} (very light grey), Lrit3^{-/-}-BC-Lrit3 (dark grey), Lrit3^{-/-}-OPL-Lrit3 (light grey) and Lrit3^{-/-}-PR-Lrit3 (black line) mice, values on the right of the row of waveforms specify the flash intensity in log cd·s/m². B) Representative photopic ERG traces at 2 months post-injection for a flash intensity of 3.0 cd·s/m². C) Average amplitude of the scotopic ERG b-wave at 2 months post-injection. D) Comparison between the average amplitude of the scotopic ERG b-wave at 2 months (filled) and 4 months (hatched) post-injection for Lrit3^{-/-}-PR-Lrit3 (black), Lrit3^{-/-}-BC-Lrit3 (dark grey) and Lrit3^{-/-}-OPL-Lrit3 mice (light grey), no statistically significant difference between 2 and 4 months for the Lrit3^{-/-}-PR-Lrit3 mice (Wilcoxon statistical test, p=0.8).

FIG. 5: ON responses in treated retinas using MEA-256 recordings.

A) Spike density function for all responsive electrodes displaying an ON component (ON only and ON-OFF) recorded on all treated retina (1 Lrit3^{-/-}-BC-Lrit3 (85 responsive electrodes), 1 Lrit3^{-/-}-OPL-Lrit3 retina, 4 Lrit3^{-/-}-PR-Lrit3 retina, 7 Lrit3^{-/-} retina and 6 Lrit3^{+/+} retina). Light stimuli is indicated as a black bar and light grey area, responses recorded at individual electrode are displayed as grey line (average of 10 repetitions), the peak firing rate amplitude and latency is overlaid with the traces as open circle. Lrit3^{+/+}recording have a different scaling (upper left) than all other conditions (middle). B) ON peak firing rate (up) and ON peak latency (bottom) for all responsive electrodes in the different conditions. Horizontal black bar represent the average value, and vertical black bar the mean+/−SD. C) Fraction of the electrode population displaying ON, ON-OFF or OFF profile of response. Unresponsive electrodes are electrodes where spontaneous activity is recorded without light-evoked spiking.

FIG. 6: Optomotor responses under both scotopic and photopic conditions

The number of head movements per minute was obtained under both scotopic and photopic conditions with spatial frequencies of 0.063 and 0.125 cycles/degree (scotopic) or 0.063 alone (photopic) for Lrit3^{-/-}-BC-Lrit3 (dark grey), Lrit3^{-/-}-OPL-Lrit3 (light grey) and Lrit3^{-/-}-PR-Lrit3 (black) mice and compared using Mann-Whitney statistical test with representative Lrit3^{+/-} (dotted black) and Lrit3^{-/-} (very light grey) mice. The star indicates a significant test (p<0.05).

### EXAMPLES

### Materials and Methods

### Ethical Statement

All animal procedures were performed according to the Council Directive 2010/63EU of the European Parliament and the Council of Sep. 22, 2010, on the protection of animals used for scientific purposes, with the National Institutes of Health guidelines and with the ARVO Statement for the Use of Animals in Ophthalmic and Vision Research. They were approved by the French Minister of National Education, Superior Education and Research (authorization delivered on Jan. 21, 2019).

### AAV Production

The production of recombinant AAVs was made by following, the plasmid cotransfection method (12). Lysates were then purified using iodixanol gradient ultracentrifugation as previously described: 4(1% iodixanol fraction was concentrated and buffer exchanged using Amicon Ultra-15 Centrifugal Filter Units (Merck Millipore, Billerica, MA, USA). Real-time PCR was used to titer the vector stocks for DNase-resistant vector genomes relatively to a standard (13).

### Intravitreal Injections

Mice were anesthetized by isoflurane inhalation (5% in oxygen for induction and 2% for maintenance). Intravitreal injections were performed at 2 (P2) or 30 (P30) days of age. Pupils were dilated (0.5% mydriaticum) and a 33-gauge needle was passed through the sclera at the ora serrata level. 1 μl of a viral stock solution at a concentration of 1.73 10¹⁴ vg/ml maximum was injected directly in the vitreous cavity.

### Electroretinogram

Mice were dark-adapted overnight before performing the ERG recordings. They were anesthetized by ketamine (80 mg/kg) and xylazine (8 mg/kg) and eye drops were used to dilate their pupils (0.5% mydriaticum 5% neosynephrine) and anesthetize the cornea (0.4% oxybuprocaine chlorohydrate). Mice corporal temperature was maintained through a heating pad along the test. Upper and lower eyelids were retracted to keep the eyes opened and bulging. Corneal lenses (Mayo Corporation, Japan) were applied on corneal surface to record the ERG. A reference electrode was placed on the nose while the ground electrode was placed above the tail. Recordings from both eyes were made in parallel. All scotopic ERG were made first using six increasing light intensity of flashes ranging from 0.003 to 30.0 cd·s/m². Each trace corresponding to one tight-intensity results from the average of five traces originating from live flashes. To ensure a saturation of rod PRs and the recording of cone-driven responses, a 10-minutes light-adaptation step at 20 cd·m² was done. Following this light-adaptation step, photopic ERGs were recorded first at 3.0 cd·s/m² and at the same intensity; 5 Hz and 10 Hz flickers were also checked. All data were analyzed with GraphPad Prism v.6 (GraphPad Software, La Jolla, CA USA)

### Optomotor Test

Optomotor test was performed as described previously (5). Mice were dark-adapted overnight before the optomotor test. Ten wild-type animals and ten knock-out animals of each lineage were studied along with the treated animals. Mice were placed on a grid platform (11.5 cm diameter, 19 cm above the bottom of the drum) at the center of a motorized drum (29 cm diameter) covered by vertical black and white stripes of a defined spatial frequency (0.063, 0.125, 0.25, 0.5 and 0.75 cycles per degree). A five minutes break was made before the test so the animal gets used to its new environment. The stripes were rotated for 1 minute clockwise and 1 minute counter-clockwise at a speed of 2 rotations per minute. An interval of 10 sec was made after the first minute. Each test was recorded with a digital infrared camera to count head movements of the mice. Tests were firstly performed under scotopic conditions then in photopic condition after 5 minutes of light adaptation (two lamps of 60 Watts). Head movements in both directions were considered to obtain the number of head movements per minute.

### MEA

After overnight dark adaptation, mice were sacrificed by CO₂ inhalation followed by cervical dislocation. Retinas were carefully dissected under dim-red light and conserved in Ames medium (Sigma-Aldrich, St. Louis, MO, USA) oxygenated with 95% oxygen and 5% CO2. Retinas were placed on a Spectra/Por membrane (Spectrum Laboratories, Rancho Dominguez, CA, USA) previously coated with poly-D-lysine and gently pressed against an MEA (MEA256 100/30 iR-ITO; Multi Channel Systems MCS, Reutlingen, Germany) using a micromanipulator, RGCs facing the electrodes. Retinas were continuously perfused with bubbled Ames medium kit 348 C at a rate of 1 to 2 ml/min and let to rest for 45 minutes before the recording session. Under dark conditions, 10 repeated full-field light stimuli at a 450 nm wavelength were applied to the samples at 4.10¹¹ photons/cm²/s for 2 seconds with 10-second interval by using a Polychrome V monochromator (Olympus, Hamburg, Germany) driven by an STG2008 stimulus generator (MCS). Raw RGC activity recorded by MEA was amplified (gain 1000-1200) and sampled at 20 kHz by using MCRack software (MCS). Resulting data were stored and filtered with a 200-Hz high-pass filter. Raster plots were obtained by using a combination of threshold detection, template matching, and cluster grouping based on principal component analysis using Spike2 v.7 software (CED Co., Cambridge, UK). Peristimulus time histograms were plotted with a bin size of 50 ms by using a custom-made script in MATLAB v.R2014b (MathWorks, Inc., Natick, MA, USA). Only RGCs with a mean spontaneous firing frequency superior to 1 Hz were considered. We subsequently determined for each sorted RGC the maximum firing frequency in an interval of 2 seconds after light onset (for ON-responses) and in an interval of 2 seconds after light offset (for OFF-responses). These values were normalized to the mean spontaneous firing frequency of the corresponding RGC. Considering that significant responses have a maximum firing frequency that is superior to the mean spontaneous firing frequency [symbol] 5 SD, we determined the time at which these significant frequencies were reached after the light onset for ON-responses and after the light offset for OFF-responses. The histograms were traced with GraphPad Prism v.6 (GraphPad Software, La Jolla, CA, USA).

Immunolocalization Studies

Animals were sacrificed by CO₂ inhalation followed by cervical dislocation. Eyes were removed and dissected to keep the posterior part of the eyes which were then fixed in ice-cold 4% paraformaldehyde for 20 minutes. Subsequently, the eye cups were washed in ice-cold PBS and cryoprotected by increasing concentrations of sucrose (ranging from 10% to 30%) in water and 0.24 M phosphate buffer for 1 hour at 4° C. for 10% sucrose and 20% sucrose solutions and overnight at 4° C. under agitation for the 30% sucrose solution. The eye-cups were then embedded in 7.5% gelatin-10% sucrose and the blocks frozen at −40° C. in isopentane and kept at −80° C. until cutting. Sections of 12 μm were generated using a cryostat (MICROM HM 560™, ThermoFisher Scientific, Waltham, MA, USA) and mounted on glass slides (Superfrost® Plus, ThermoFisher Scientific). Mouse retina sections were treated to decrease background noise (Antigen Retrieval Reagent, Biotechne, Minneapolis, MN, USA) for 4 minuses at 92° C. and subsequently blocked for 1 hour at room temperature in PBS1X 10% Donkey Serum (v/v), 0.1% Triton X-100. Primary antibodies and the dilutions used were: rabbit anti-LRIT3 (1:200, Neuillé et al., 2015) and sheep anti-TRPM1 (1:500;Cao et al). The sections were incubated with primary antibodies diluted in PBS1X 2% Donkey Serum (v/v), 0.1% Triton X-100 for 1 hour at room temperature. After washes with PBS1X 0.1% Triton-X100, the sections were incubated with anti-rabbit and anti-sheep secondary antibodies coupled with Alexa Fluor 488, or Cy3 (Jackson ImmunoReserach) along with 4′,6-diamidino-2-phenylindole (DAPI), all used at 1:1000, for 0.5 hours at room temperature. Subsequently, the sections were cover-slipped with mounting medium (Mowiol, Merck Millipore, Billerica, MA, USA). Fluorescence images retinal sections were acquired with a confocal microscope (FV1000, Olympus). Images for figures were handled with the Image J software (ImageJ Software).

### Results

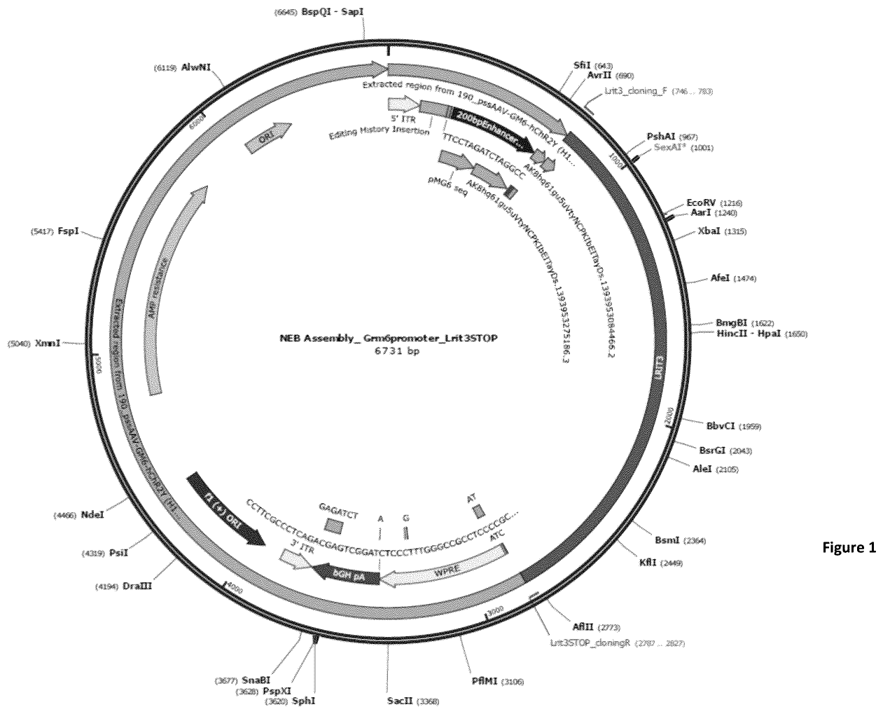

Immunolocalization studies in human and mouse retina showed LRTI3 protein in the outer plexiform layer (OPL). If it still undefined whether this localization is postsynaptic at the dendritic tips of ON-bipolar cells (BCs) or/and presynaptic at the synapse of photoreceptors. Herein, to rescue the phenotype in the nob6 mouse model (referred later as Lrit3^{-/-}), two different constructs were designed using two different promoters: the Grm6 promoter that has been shown to drive expression of the transgene specifically to ON-Bipolar cells and the GRK1 promoter which promotes expression in both, rod and cone photoreceptors. These two constructs were encapsidated in the AAV2-7m8 serotype and either injected alone or mixed at a 1:1 ratio (FIG. 1). Mice injected with the Grm6 promoter construct will be noted as Lrit3^{-/-}-BC-Lrit3, mice treated with the GRK promoter construct will be referred as Lrit3^{-/-}-PR-Lrit3 and mice injected with both constructs will be named Lrit3^{-/-}-OPL-Lrit3.

### Transgene Expression Rescues LRIT3 Production and Localization

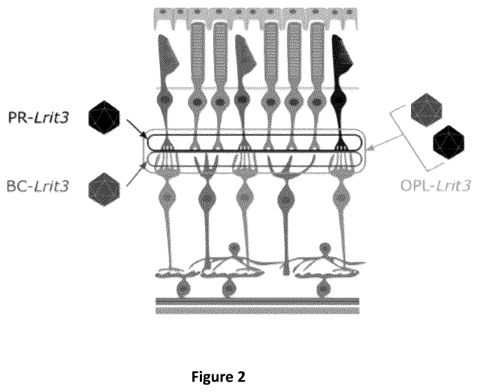

In the Lrit3^{-/-} mice, LRIT3 production is abolished in the OPL in both rod-to-rod BC (FIG. 2A, arrows) and cone-to-cone BC synapses (FIG. 2A, arrow heads) ((7)). The restoration and proper localization of LRIT3 following treatment was investigated through immunolocalization studies. Lrit3^{-/-}-BC-Lrit3 retinas treated at P30 displayed LRIT3 in the OPL which is absent in untreated Lrit3^{-/-} retinas. LRIT3 appeared in both rod-to-rod ON-bipolar cell synapses (punctate staining, arrows) along with cone-to-cone ON-BCs synapses (line-like staining, arrow heads) (FIG. 2A).

### TRPM1 Relocalized at the Dendritic Tips of ON-Bipolar Cells Following Treatment

TRPM1 localization at the dendritic tips of ON-BCs is essential to ensure a correct signaling through ON-BCs. In the Lrit3^{-/-} mouse model, dendritic tip localization of TRPM1 is abolished, while it is still present within BCs ((7)). The localization of TRPM1 partners following treatment in either photoreceptors or ON-BCs was investigated through immunolocalization studies. Restoration of TRPM1 localization in presumed dendritic tips of ON-BCs is shown in FIG. 2B (arrows).

### AAV-Mediated LRIT3 Expression in the OPL Restores the ERG B-Wave

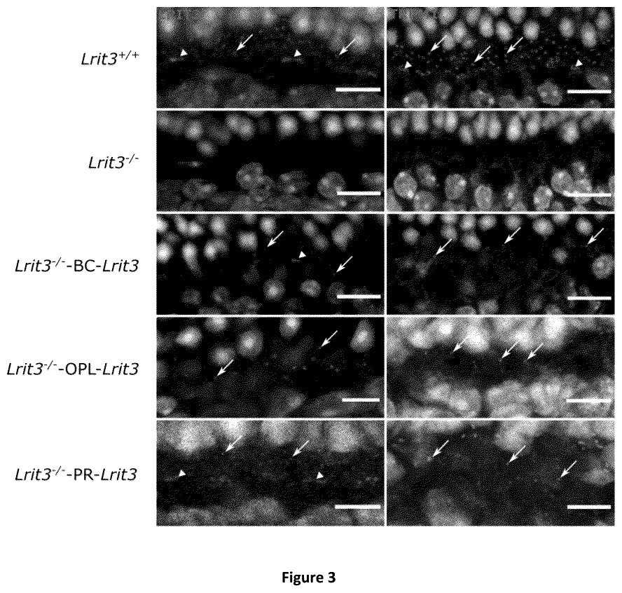

In Lrit3^{-/-} animals, the transmission of the visual signal between photoreceptors and ON-BCs is disrupted as shown by the absence of the b-wave on the ERG under scotopic and photopic conditions ((5)). ERG recordings performed two months after treatment on Lrit3^{-/-}-BC-Lrit3mice injected at P30 revealed a partial rescue of the b-wave under scotopic conditions (FIG. 3) with highest restoration at the lowest flash intensity. The amplitude of the b-wave corresponded to a rescue of 45% of the b-wave amplitudes from Lrit3^{+/+} mice. No improvement of the photopic b-wave was recorded in the Lrit3^{-/-} treated mice. However, these results were obtained in a non-significant number of Lrit3^{-/-}-BC-Lrit3 mice. Similarly, ERG recordings in Lrit3^{-/-}-OPL-Lrit3 mice treated at P30 also presented a b-wave which amplitude corresponded to 45% of Lrit3+/+ mouse amplitudes at the lowest light intensity. As Lrit3^{-/-}-BC-Lrit3 mice, the scotopic b-wave was the highest at low flash-intensities (FIG. 3) and the restoration was only observed under scotopic conditions. Similarly, only few mice functionally responded to the treatment. Strikingly, partial restoration was obtained in Lrit3^{-/-}-PR-Lrit3 animals (N=4) treated at P30 under scotopic conditions. The amplitude of the b-wave was rescued at 58% of the b-wave amplitude of Lrit3^{+/+} mouse amplitudes, but again only for scotopic conditions. This restoration will be followed up at least 4-month post-injection.

### ON Bipolar Cells Signaling Pathway Restoration Recovers ON-Responses in RGCs

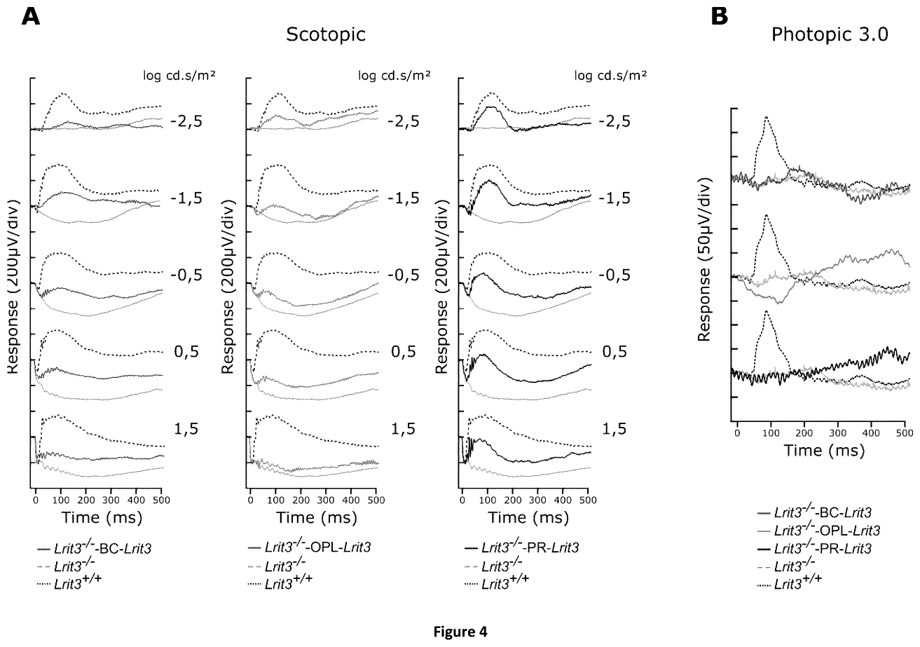

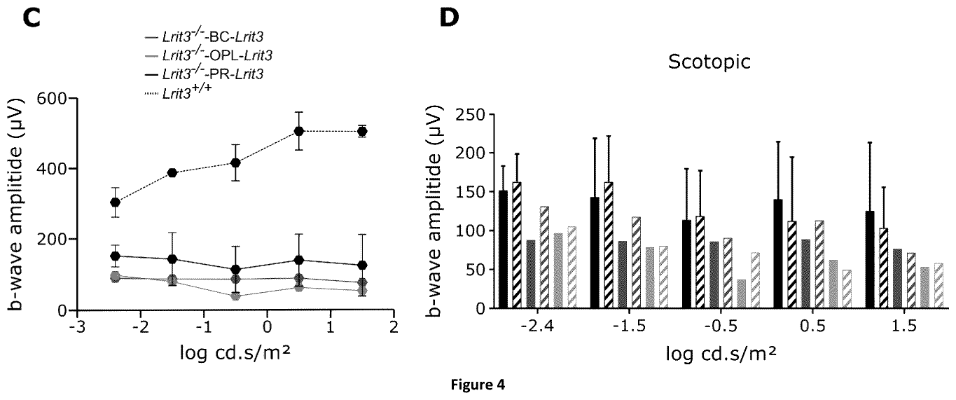

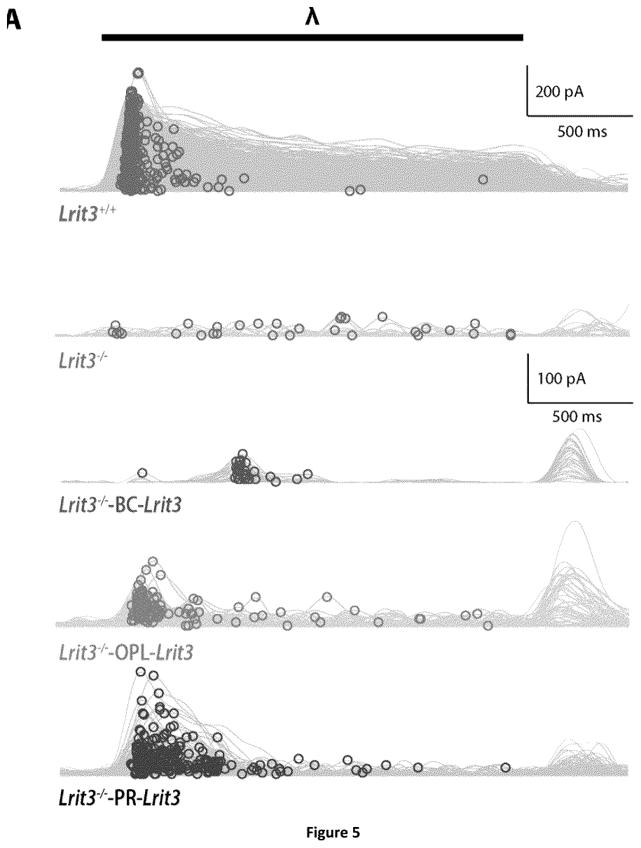

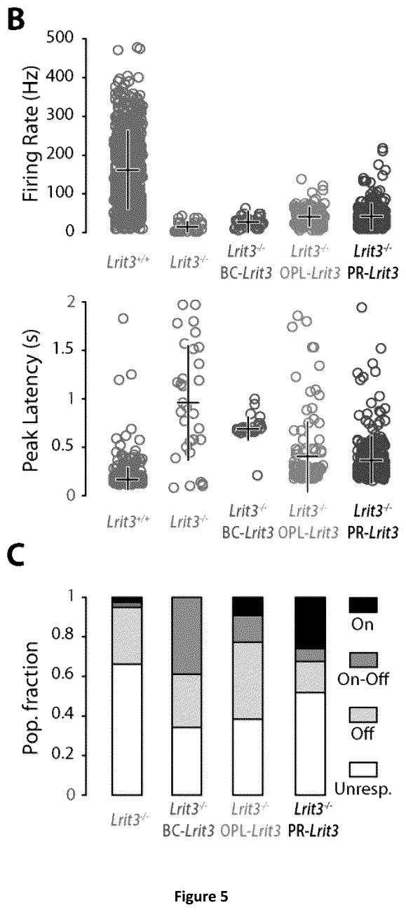

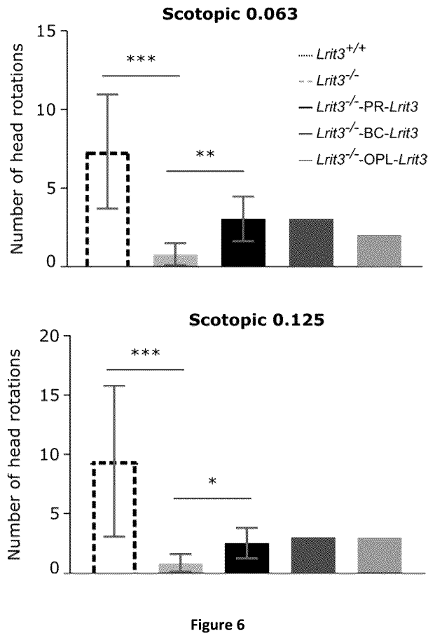

As previously described, ON responses are also abolished at the level of retinal ganglion cells (RGCs) in Lrit3^{-/-} mice ((6)). LRIT3 correct localization is mandatory for TRPM1 localization and function and therefore for the further propagation of the visual signal towards ON-ganglion cells. To confirm the functional rescue following treatment, light-evoked ON-RGCs responses were recorded through multi-electrode array (MEA) from Lrit3^{-/-}-BC-Lrit3, Lrit3−/−-OPL-Lrit3 and Lrit3−/−-PR-Lrit3 retinas. When stimulated, RGCs from Lrit3^{-/-}-BC-Lrit3 retinas presented delayed ON responses (dON) that spiked 1 s after light onset on a light stimulation of 2 seconds (grey square), and OFF responses while no ON-responses were recorded in untreated Lrit3^{-/-} retinas (FIG. 4, ON-responses dark arrow, OFF-responses blue arrow). Furthermore, when the retina was perfused with L-AP4 an agonist of mGluR6/GRM6, these responses were abolished and subsequently dON were again recordable after the L-AP4 washout. In Lrit3^{-/-}-Lrit3 retinas, ON-responses were also noted but they were not delayed as RGCs spiked a few milliseconds after light onset. As observed with Lrit3^{-/-}-BC-Lrit3, L-AP4 perfusion abolished these responses which were again detectable after washout.

### LRIT3 Partial Rescue Improved Optomotor Response in Treated Mice

It has been shown that the transmission defect between photoreceptors and ON-bipolar cells in the Lrit3−/− mouse has an impact on the visual perception of these mice ((5)). To investigate if the partial functional rescue observed with ERG recordings and on MEA at the level of RGCs, optomotor responses of the treated mice were studied. For the two types of treated mice, Lrit3^{-/-}-BC-Lrit3 and Lrit3^{-/-}-OPL-Lrit3, optomotor reflexes seem to be improved compared to untreated Lrit3^{-/-} mice (FIG. 5).

### Conclusion

Together these findings indicate that adult mice with cCSNB can he treated with gene replacement using an AAV-based approach targeting BCs photoreceptor cells. This approach results in a partial rescue of scotopic vision in cCSNB mice similarly altered in patients with cCSNB. The photopic vision could not be restored in the cCSNB mouse model, but the phenotype in cCSNB mice is also strikingly more severely affected than the one in patients with cCSNB. Thus, it will be important to treat larger animals, e.g. dogs lacking the same gene with a more similar phenotype to validate the gene therapy approach prior to treatment of cCSNB patients.

### REFERENCES

1. Zeitz C, Robson A G, Audo I. Congenital stationary night blindness: an analysis and update of genotype-phenotype correlations and pathogenic mechanisms. Progress in retinal and eye research. 2015;45:58-110.
2. Bellone R R, Brooks S A, Sandmeyer L, Murphy B A, Forsyth G, Archer S, et al. Differential gene expression of TPRM1, the potential cause of congenital stationary night blindness and coat spotting patterns (LP) in the Appaloosa horse (Equus caballus). Genetics, 2008;179(4):1861-70.
3. Das R G, Becker D, Jagannathan V, Goldstein O, Santana E, Carlin K, et al. Genome-wide association study and whole-genome sequencing identify a deletion in LRIT3 associated with canine congenital stationary night blindness. Scientific reports. 2019;9(1):14166.
4. Zeitz C, Jacobson S G, Hamel C P, Bujakowska K, Neuille M, Orhan E, et al. Whole-exome sequencing identifies LRIT3 mutations as a cause of autosomal-recessive complete congenital stationary night blindness. American journal of human genetics. 2013;92(1):67-75.
5. Neuille M, El Shamieh S, Orhan E, Michiels C, Antonio A, Lancelot M E, et al. Lrit3 deficient mouse (nob6): a novel model of complete congenital stationary night blindness (cCSNB). PloS one. 2014;9(3)e90342.
6. Neuille M, Cao Y, Caplette R, Guerrero-Given D, Thomas C, Kamasawa N, et al. LRIT3 Differentially Affects Connectivity and Synaptic Transmission of Cones to ON- and OFF-Bipolar Cells. Investigative ophthalmology & visual science. 2017;58(3):1768-78.
7. Neuille M, Morgans C W, Cao Y, Orhan E, Michiels C, Sahel J A, et al. LRIT3 is essential to localize TPRM1 to the dendritic tips of depolarizing bipolar cells and may play a role in cone synapse formation. Invest Ophthalmol Vis Sci. 2015:ARVO E-Abstract 2612.
8. Hasan N, Pangeni G, Ray T A, Fransen K M, Noel J, Borghuis B G, et al. LRIT3 is required for Nyctalopin expression and normal ON and OFF pathway signaling in the retina. eNeuro. 2020.
9. Trapani I, Auricchio A. Seeing the Light after 25 Years of Retinal Gene Therapy. Trends in molecular medicine. 2018;24(8):669-81.
10. Scalabrino M L, Boye S L, Fransen K M, Noel J M, Dyka F M, Min S H, et al. Intravitreal delivery of a novel AAV vector targets ON bipolar cells and restores visual function in a mouse model of complete congenital stationary night blindness. Human molecular genetics. 2015;24(21):6229-39.
11. Hasan N, Pangeni G, Cobb C A, Ray T A, Nertesheim E R, Ertel K J, et al. Presynaptic Expression of LRIT3 Transsynaptically Organizes the Postsynaptic Glutamate Signaling Complex Containing TRPM1. Cell reports. 2019;27(11):3107-16 e3.
12. Choi V W, Asokan A, Haberman R A, Samulski R J. Production of recombinant adeno-associated viral vectors for in vitro and in vivo use. Current protocols in molecular biology. 2007:Chapter 16:Unit 16 25.
13. Aurnhammer C, Haase M, Muether N, Hausl M, Rauschhuber C, Huber I, et al. Universal real-time PCR for the detection and quantification of adeno-associated virus serotype 2-derived inverted terminal repeat sequences. Human gene therapy methods. 2012;23(1):18-28.

1. An expression cassette comprising (i) GRK1 promoter and/or a 200 bp enhancer of the GRM6 promoter fused to the SV40 promoter (GRM6 promoter) and (ii) a nucleic acid sequence encoding LRIT3 that is operably linked to said promoter.

2. The expression cassette of claim 1, wherein said nucleic acid sequence encoding human LRIT3 has at least 90% identity with SEQ. ID. NO:1.

3. The expression cassette of claim 1, comprising only a GRK1 promoter, or only a GRM6 promoter or both GRK1 promoter and GRM6 promoter.

4. A recombinant expression vector comprising the expression cassette according to claim 1.

5. A recombinant adeno-associated virus (AAV) vector comprising:
an AAV capsid;
an expression cassette according to claim 1.

6. The recombinant AAV vector of claim 5, wherein the AAV capsid is AAV2.7m8.

7. The recombinant AAV vector of claim 5, wherein it also contains woodchuck hepatitis posttranscriptional regulatory element (WPRE) and/or poly-Adenine (polyA).

8. The recombinant AAV vector of claim 5, wherein it expresses LRIT3 in photoreceptor cells.

9. The recombinant AAV vector of claim 5, for use as a medicament.

10. The recombinant AAV vector of claim 5, for use in the treatment of congenital stationary night blindness (CSNB).

11. The recombinant AAV vector of claim 5, for use in the treatment of CSNB, wherein CSNB is complete congenital stationary night blindness (cCSNB).

12. The recombinant AAV vector of claim 5, for use in the treatment of CSNB or cCSNB, wherein the amount of recombinant AAV vector administered is sufficient to provide a therapeutic effect to said mammal.

13. A pharmaceutical composition comprising a recombinant AAV vector of claim 5 and a pharmaceutical acceptable carrier.

14. A pharmaceutical composition according to claim 13, comprising a recombinant AAV vector of claim 5 comprising the GRK1 promoter or comprising the GRM6 promoter.

15. The pharmaceutical composition of claim 13, for use in the treatment of CSNB.

16. The pharmaceutical composition of claim 13, for the treatment of CSNB, wherein said composition is for intravitreal or subretinal administration.

17. A method of treating CSNB in a subject, said method comprising administering to said subject an effective concentration of the pharmaceutical composition of claim 13.
